(19) United States
(12) Patent Application Publication
(10) Pub. No.: US 2023/0069765 A1
(43) Pub. Date: Mar. 2, 2023

(54) CONNECTOR IMPLANT FOR CONNECTING TWO POSTERIOR ROD PORTIONS

(71) Applicant: INNO4SPINE AG, Stans (CH)

(72) Inventors: Tamás Fülöp FEKETE, Zürich (CH); Heiko KOLLER, Kufstein (AT); Dezsö János JESZENSZKY, Küsnacht (CH); Daniel HASCHTMANN, Küsnacht (CH); Adriano VIGANÒ, Pfäffikon (CH); Tom OVERES, Biberist (CH)

(73) Assignee: INNO4SPINE AG, Stans (CH)

(21) Appl. No.: 17/801,204

(22) PCT Filed: Jan. 24, 2021
(86) PCT No.: PCT/IB2021/050527

§ 371 Date: Aug. 19, 2022

(30) Foreign Application Priority Data
Feb. 21, 2020 (CH) 00208/20

(51) Int. Cl. A61B 17/70

(52) U.S. Cl. CPC A61B 17/705; A61B 17/7083

(57) ABSTRACT

A spinal rod connector assembly is provided for connecting and axially aligning two in situ spinal posterior rod portions. The spinal rod connector assembly comprises a rod connector and one or more rod fasteners. The rod connector comprises a rod receiving passage extending between two opposite ends of the rod connector. Seats are provided along the passage to engage with the rod portions when the spinal rod assembly has been implanted. The rod connector comprises one or more locking holes extending from the connector top side into or towards the rod receiving passage. The one or more locking holes being sized and shaped for engaging with the one or more rod fasteners.

### TECHNICAL FIELD OF THE INVENTION

The present invention relates to a spinal rod connector assembly for axially aligning a first broken rod portion and a second broken rod portion of a broken in situ spinal posterior rod system, and for connecting the rod portions to each other. The connector assembly may be placed between two bone fasteners, most commonly pedicle screws, but may also span over one bone fastener. The invention also relates to a method for connecting broken spinal rod portions by means of a top-loading approach and a side-loading approach. The invention further relates to a kit containing a plurality of rod connectors enabling spinal rod portions which are straight, curved or angled relative to each other to be connected. The invention equally relates to a kit of rod connectors, which are mirror-imaged, and allow the rod portions that are shifted relative to each other to be realigned, in a minimally invasive manner, by using the rod connector itself.

### BACKGROUND OF THE INVENTION

In orthopaedic surgery around the spine, posterior spinal stabilisation systems are often placed to a target site to realign, re-correct or stabilise the spinal column to compensate for malalignment caused by for example degeneration of the spine, born malalignments, such as excessive lordosis, kyphosis and scoliosis, and for example trauma, such as fractures. During the fusion phase of the spine, during which the realigned vertebral bodies grow together, primary stability to the spine is provided by the posterior spinal stabilisation system in combination with an interbody fusion device. During this phase, the posterior spinal stabilisation system needs to withstand high loads and stresses. These loads may exceed the strength of the posterior spinal stabilisation system and cause its fracture. Most often the system fails due to fracture of the posterior rod, that is connecting at least two bone fasteners. Two fracture scenarios are most common, namely a fracture directly next to a bone fastener, at the interface of the bone fastener and the rod, and a fracture in the middle between two bone fasteners. The first scenario is characterised by a very short fractured rod portion and a long fractured rod portion between the bone fasteners. The second scenario is characterised by having two substantially equally long rod ends between the bone fasteners.

A construct, such as a posterior spinal stabilisation system, is best repaired in the original orientation, re-establishing the original correction. To be able to extend the system, according to common techniques, the full in situ rod is removed from the bone fasteners, and replaced by a new rod. Removal is executed by unscrewing so-called setscrews that rigidly fix the rod into the tulip head of the bone fastener. The rod is then pulled out after removal of all the setscrews. Now, a new rod is attached to the bone fasteners that are in place. The removal of the broken rod destabilises the spine during surgery, and requires a new, often large skin and soft tissue access to the implants. Especially in the cases of longer constructs, this can be a very invasive surgery.

### SUMMARY OF THE INVENTION

It is an object of the present invention to overcome at least some of the problems associated with replacing or repairing a broken pedicle screw and rod construct. A solution is needed that may obviate the need of removal and replacement of the broken rod, and that also provides the ability to re-stabilise the broken spinal posterior rod system in a minimally invasive manner. Furthermore, a solution is also needed for different rod breakage scenarios, where the first scenario is characterised by a very short fractured rod end and a long fractured rod end between the bone fasteners, and the second scenario is characterised by having two substantially equally long rod ends between the bone fasteners. Moreover, there is a need for a solution which forms a spinal rod connector, and which allows different systems of different suppliers, and/or bent rods, straight rods, and/or curved rods to be rigidly repaired.

According to a first aspect of the invention, there is provided a spinal rod connector assembly as recited in claim 1.

The proposed spinal rod connector assembly has for instance the advantage that it helps a surgeon to substantially repair an existing and broken in situ spinal rod without the necessity of removing the rod portions and thus without destabilizing the spine and without requiring a large incision to replace the broken rod by a new rod.

According to a second aspect of the invention there is provided a kit of rod connectors as recited in claim 17.

The proposed kit has for instance the advantage that it helps the surgeon to repair an existing in situ spinal rod, where the original rod per repair situation may have e.g. any of the following shapes: a bent, curved or straight shape. The kit contains at least a first rod connector and a second rod connector, where the first rod connector allows straight rod portions to be connected, and the second rod connector allows rod portions of a curved shape to be connected.

According to a third aspect of the invention there is provided a method of repairing an in situ construct as recited in claim 20.

The proposed method allows a first rod portion to be connected to a second rod portion, in a top-loading manner, and allows the rod portions that are shifted with respect to each other to be realigned in a minimally invasive manner, by using the rod connector itself.

Other aspects of the invention are recited in the dependent claims attached hereto.

### BRIEF DESCRIPTION OF THE DRAWINGS

Other features and advantages of the invention will become apparent from the following description of non-limiting example embodiments, with reference to the appended drawings, in which:

FIGS. 1A to 1C depict in different perspective views an example rod connector assembly according to an embodiment of the present invention;

FIGS. 2A to 2D depict in different views a variant of the rod connector body;

FIG. 3 depicts a rod fastener in detail;

FIGS. 4A to 4C depict in different perspective views a bent variant of the rod connector body;

FIGS. 5A to 5C depict in different perspective views a curved variant of the rod connector body;

FIGS. 6A to 6N illustrate the surgical steps to repair a broken rod by using a top-loading approach;

FIGS. 7A to 7C depict in different perspective views another variant of the rod connector body, where the body is sized and shaped for a side-loading approach;

FIGS. 8A to 8C depict in perspective views a bent variant of the rod connector body of FIGS. 7A to 7C;

FIGS. 9A to 9C depict in perspective views a curved variant of the rod connector body of FIGS. 7A to 7C;

FIGS. 10A and 10B depict the surgical steps to repair a broken rod by using a side-loading approach;

FIGS. 11A to 11D depict a variant of the rod connector assembly, where an intermediate insert is configured to engage against the broken rod portions;

FIGS. 12A and 12B depict a variant of the rod connector used to repair a broken rod, where the rod connector is configured to bridge over a rod fastener;

FIG. 12C depicts a rod fastener to be used with the rod connector of FIGS. 12A to 12B;

FIGS. 13A to 13C depict the surgical steps to repair a broken rod by using the rod connector of FIGS. 12A and 12B by using a side-loading approach; and

FIGS. 14A to 14D depict the surgical steps to repair a broken rod using the rod connector of FIGS. 12A and 12B by using a top-loading approach.

### DETAILED DESCRIPTION OF THE INVENTION

The embodiments of the present invention will now be described in detail with reference to the attached figures. The embodiments are described in the context of repairing an in situ posterior spinal construct having a broken rod. Although the invention is specifically described in the context of extending the posterior spinal construct, the teachings of the invention are not limited to this environment. The teachings of the present invention are equally applicable to repairing rod-based stabilisation constructs for other bones as well. Identical or corresponding functional and structural elements which appear in the different drawings are assigned the same reference numerals. When the words first and second are used to refer to different elements, it is to be understood that this does not necessarily imply or mean that the first and second elements are somehow structurally substantially different elements or that their dimensions are substantially different unless specifically stated.

One example way of repairing an in situ spinal construct is characterised by substantially equally long rod ends between two adjacent bone fasteners, where the ends have a sufficient length for attachment of a connector thereto. A connector can be connected between the two adjacent bone fasteners. Another example way of repairing an in situ spinal construct is characterised by very short and long fractured rod ends between the bone fasteners, where the short end is too short for attachment of a connector thereto. The connector must be connected over the bone fastener at the opposite side of the bone fastener. A bone fastener in this context means an intermediate structural element, which can be brought into the target bone, and forms a stabile connection between the target bone and the remaining spinal construct. Most often a bone fastener is a fastening element, such as a screw.

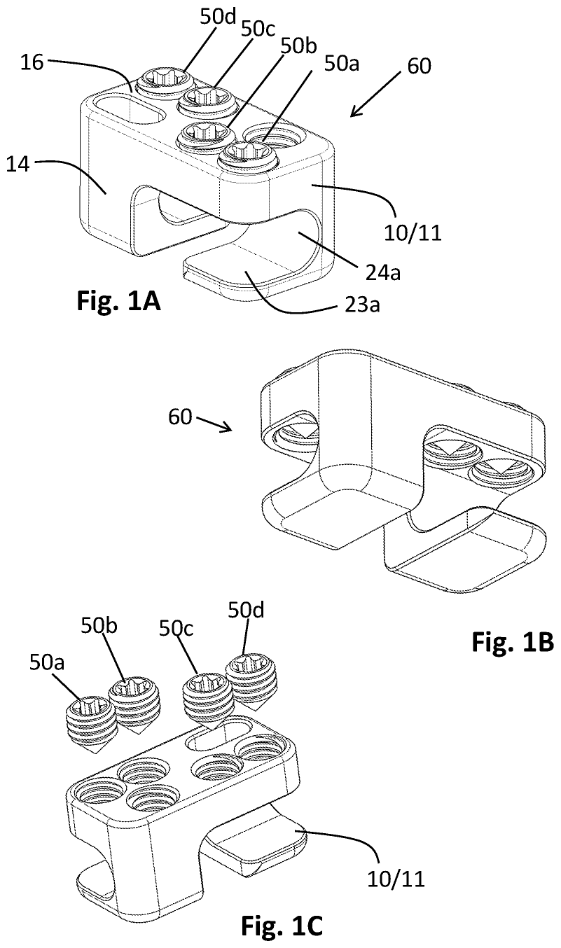

FIGS. 1A and 1B depict a rod connector assembly 60 according to a first embodiment in perspective views in an assembled configuration. FIG. 1C depicts the rod connector assembly 60 according to the first embodiment in a disassembled configuration. In this example embodiment, the rod connector assembly 60 comprises a rod connector 10 and at least one rod fastener 50a, 50b, 50c, 50d. The rod connector 10 comprises one monolithic body 11, i.e., a body constructed from a single piece. In one embodiment, as explained in greater detail later, the rod fasteners are pre-assembled within the rod connector.

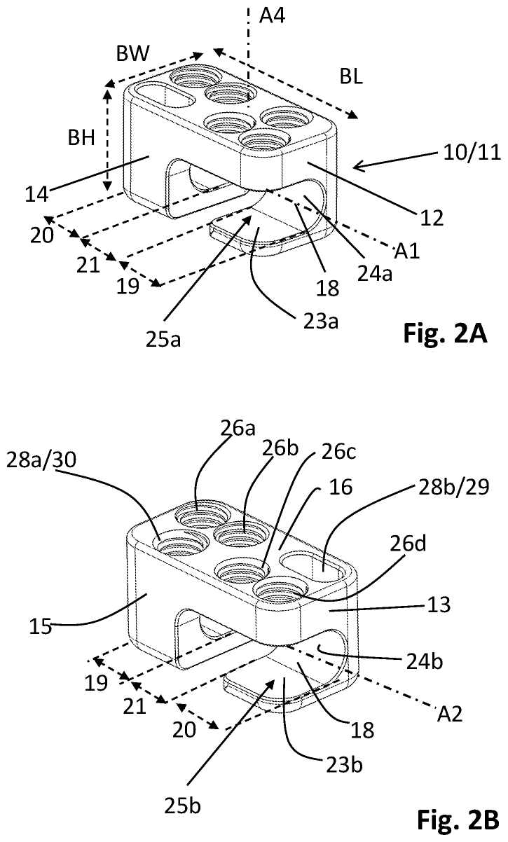

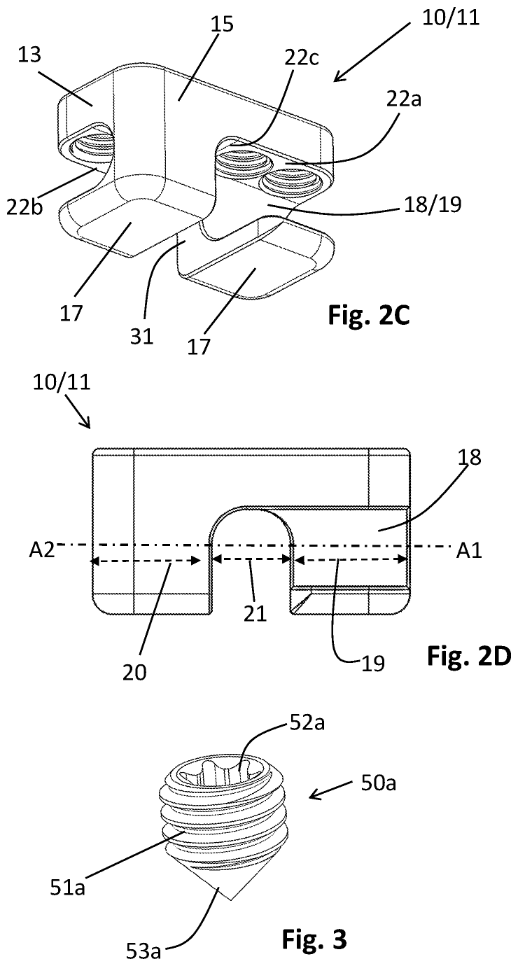

Referring to FIGS. 2A to 2D, the example rod connector 10 is shown in detail. The rod connector 10 comprises a rod connector body 11 having a body length BL, a body width BW and a body height BH, where the body width BW and body height BH define a first body end 12 (also referred to as a first connector end) and a second body end 13 (also referred to as a second connector end), while the body length BL and body height BH define a first body side 14 (also referred to as a first connector side) and a second body side 15 (also referred to as a second connector side), and the body length BL and body width BW define a body top side 16 (also referred to as a connector top side) and a body bottom side 17 (also referred to as a connector bottom side). The first and second body ends 12, 13 are thus located longitudinally at the opposing ends of the connector body 11, while the first body side 14 and the second body side 15 are located at the opposing sides of the body 11, and the body top side 16 and the body bottom side 17 are also located at the opposing sides of the body 11. It is to be understood that for illustrative purposes, the connector body is depicted as being substantially block shaped with rounded edges. Alternatively, the body may have for example a cylindrical, tubular, oblong, or triangular shape, or it may be differently shaped, but it would still have an average width, length and height defining a physical or virtual first body end 12, a second body end 13, a first body side 14, a second body side 15, a body top side 16 and a body bottom side 17.

The rod connector body 11 further comprises a rod receiving passage 18 extending between the first body end 12 and the second body end 13, where the passage 18 comprises a first passage end section 19, a second passage end section 20 and a passage middle section 21 located between the first and second passage end sections. These different passage sections are thus located adjacently and longitudinally along the passage. The first passage end section is at least open or extending into the first body end 12 and into the first body side 14. In a similar manner, the second passage end section is at least open or extending into the second body end 13 and the second body side 15. In other words, in this specific design of the rod connector, the first passage end section and the second passage end section open up in opposite directions. Thus, these end sections are arranged and located opposite to each other.

The first passage end section comprises a first passage top wall 22a, a first and substantially opposite first passage bottom seat 23a, and a first passage side seat 24a extending between the top wall and the bottom seat. Opposite to the first passage side seat 24a, a first passage side entrance 25a is present. In a similar manner, the second passage end section comprises a second passage top wall 22b, a second and substantially opposite second passage bottom seat 23b, and a second passage side seat 24b extending between the top wall and the bottom seat. Opposite to the second passage side seat 24b, a second passage side entrance 25b is present. The passage sections are sized and shaped to capture or receive (for instance snugly or play-free manner) the rod portions as described later. In the present example, the passage middle section 21 is sized and shaped as a first pocket 31 extending at the body bottom side 17 over the full body width BW. Therefore, the passage middle section substantially only comprises a third passage top wall 22c. Furthermore, the first passage end section and the second passage end section define a first passage central axis A1, and a second passage central axis A2, respectively, which in this example are substantially parallel to the longitudinal axis of the rod connector. These axes are aligned and they coincide with each other, or they intersect each other. As explained in greater detail later, the first pocket 31 forms a cut-out, which can be engaged over at least one rod portion in a top-loading approach. Then, upon turning the rod connector around its axis A4, the rod portions are simultaneously captured within the passage end sections. In other words, the first passage side seat 24a engages with a first side of one of the rod portions, while the second passage side seat 24b engages with a second, and opposite side of the other rod portion. Simultaneously, the passage top walls and the bottom seats also engage with the rod portions so that the rod portions can be snugly captured within the passage 18. It is to be noted that in the examples explained in the present description, the top walls and the bottom seats have a flat or substantially flat surface. Furthermore, in the examples explained in the present description, the top wall surfaces and the bottom seat surfaces are parallel or substantially parallel to each other, although they could have given angle between them instead. Moreover, in the example shown, the bottom seats extend from their respective side wall until the respective body side wall.

Referring to the body top side 16 of the rod connector, the body top side 16 comprises at least one locking hole 26a, 26b, 26c, 26d, extending from the body top side into or over the passage. In this example, the rod connector comprises four locking holes. The locking holes are sized and shaped to engage with at least one of the rod fastener 50a, 50b, 50c, 50d (in this example first to fourth rod fasteners). According to one example, at least one of the locking holes comprises an internal thread feature (female thread), and at least one of the rod fasteners comprises an external thread feature (male thread), or vice versa. Furthermore, the body top side 16 may comprise at least one first instrument attachment means 28a, 28b, such as a hole or recess (threaded or non-threaded) for connecting to an implant inserter or insertion instrument 70, as explained in greater detail later. Alternatively (not depicted) at least one the first body end 12, second body end 13, first body side 14, second body side 15, and body bottom side 17 comprises at least one instrument attachment means 28a, 28b. In a preferred embodiment, for the rod connector as depicted, being designed for a top-loading surgical approach, the attachment means is located on the top side, thereby keeping the implant and instrument assembly as small as possible, and thereby providing ease of disengagement of both the inserter and implant when implanted and secured in its final position. According to another example, the first attachment means 28a is sized and shaped as a threaded bore 30, and the second attachment means 28b is sized and shaped as a (non-threaded) recess 29. The attachment means are sized and shaped to engage with instrument features that are shaped in a substantially complementary manner and rigidly fixate the rod connector assembly to the insertion instrument 70, and thereby allow forceful manipulation of the rod connector. It is to be noted that the attachment means may be compatible with various state-of-the-art connection methods.

When a posterior spinal construct is implanted, often in certain sections, the rods need to be adapted or contoured to the intended anatomical correction. This adaption is made by bending or reshaping the rod. Alternatively, pre-bent rods can be obtained. A bend can be a hard bend, comprising a relatively short bending area or region between for example two straight areas or regions. Alternatively, a soft bend can be made, wherein a relatively constant curvature is given to the rod.

Referring to FIG. 3, one of the rod fasteners 50a is shown in greater detail. The rod fastener 50a comprises a first external locking feature or means 51a, and a first drive 52a. The first drive 52a is intended to engage with a locking tool such as a screw driver, providing a means to lock the rod fastener into the rod connector. In this example, the first external locking means is shaped as an external thread. Furthermore, in this example, the rod fastener comprises a conical tip 53a. The conical tip is sized and shaped to engage eccentrically against a rod portion. In this manner, the rod portion can be pressed downwards and sideways into the side and bottom seat of a passage section. Alternatively, the rod fastener may comprise a flat end or tip. In the present example, the first and at least the second rod fasteners 50a, 50b are substantially identical elements.

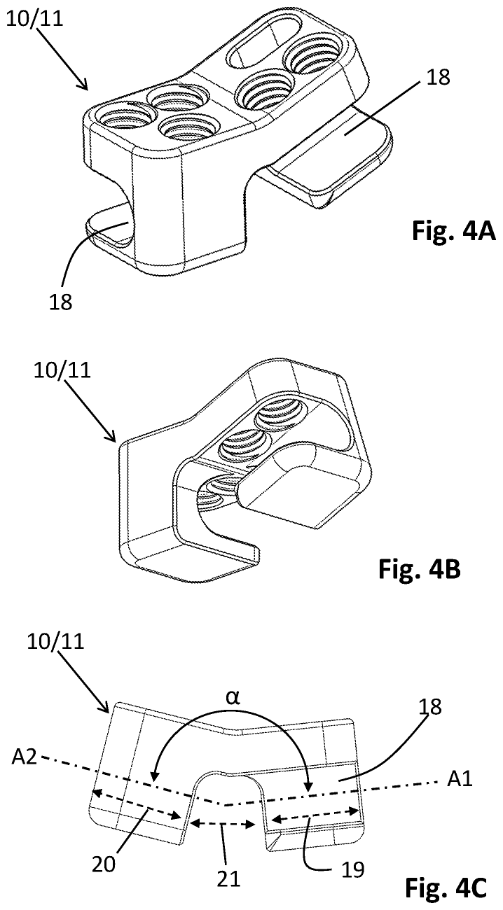

With reference to FIGS. 4A to 4C, an alternative design or shape of the rod connector is shown, addressing the need to repair a fractured rod, broken in a hard bend region. Therefore the first passage end section 19 and the second passage end section 20 are oriented at an obtuse angle α in relative to each other. In this example, the rod connector is curved along one dimension, but it could instead be curved along at least two orthogonal dimensions. In this specific example, the rod connector 10 is curved along the surface defined by the body top side 16 (or the body bottom side).

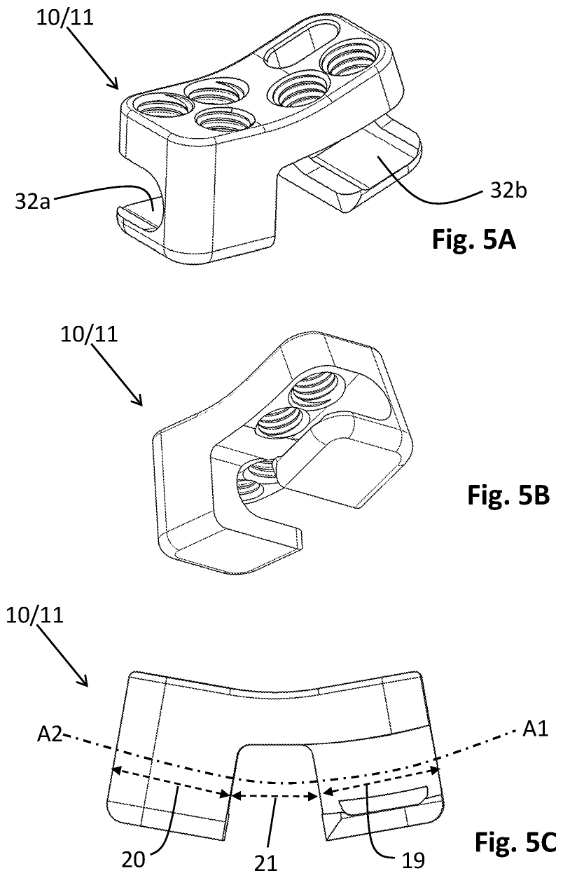

With reference to FIGS. 5A to 5C, an alternative design or shape of the rod connector is shown, addressing the need to repair a fractured rod, broken in a soft bend or curved region. Therefore the first passage end section 19 and the second passage end section 20 have a curvature of equal radius as the rod they are intended to receive within the passage section. It is to be noted that next to straight, angled or curved rod connectors, also combinations of different passage sections can be created, for example so that a curved passage section extends into a straight passage section. The curvature direction(s) may also in this case follow the principles explained with reference to FIGS. 4A to 4C.

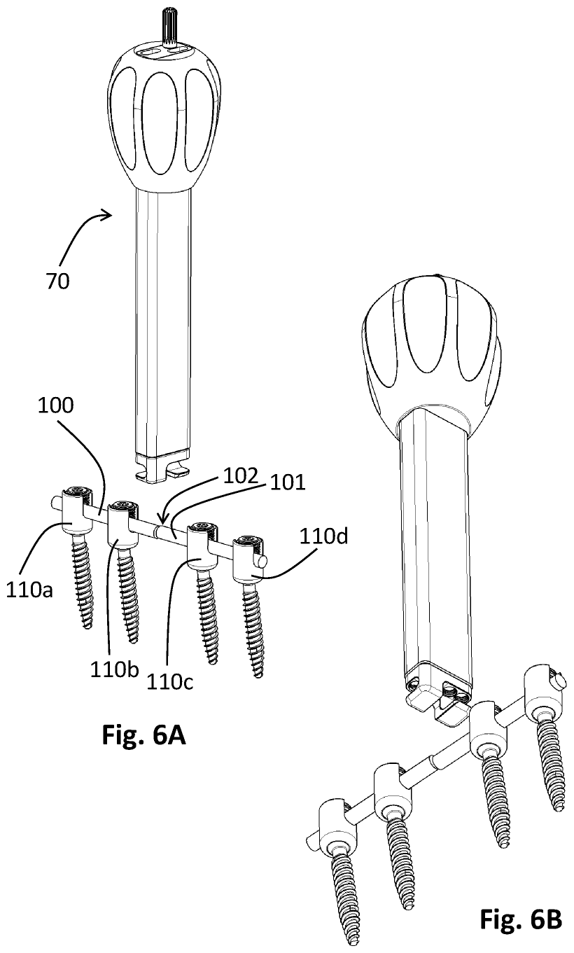

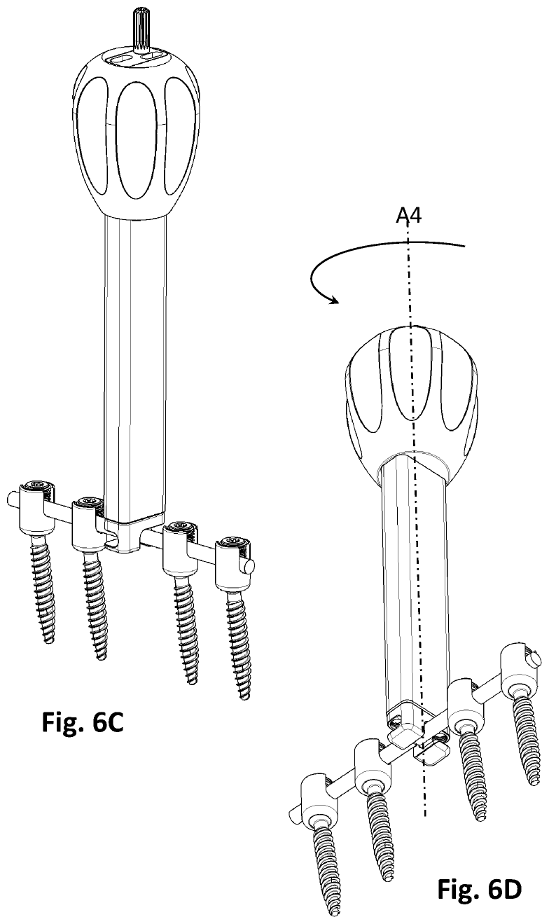

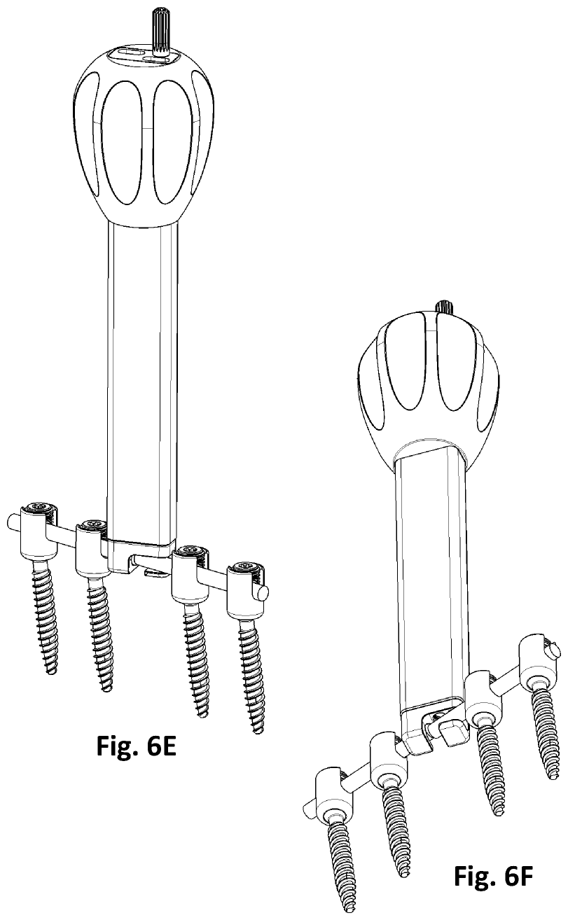

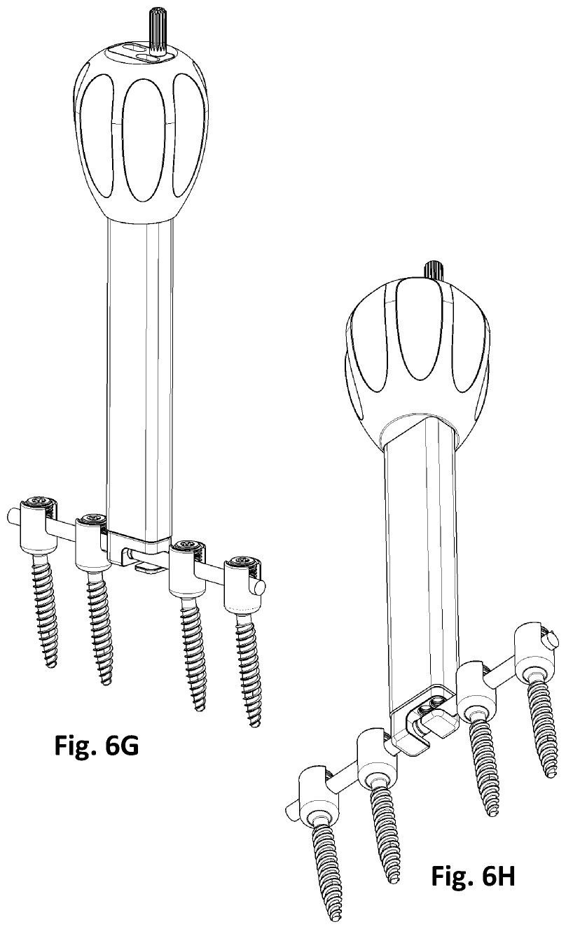

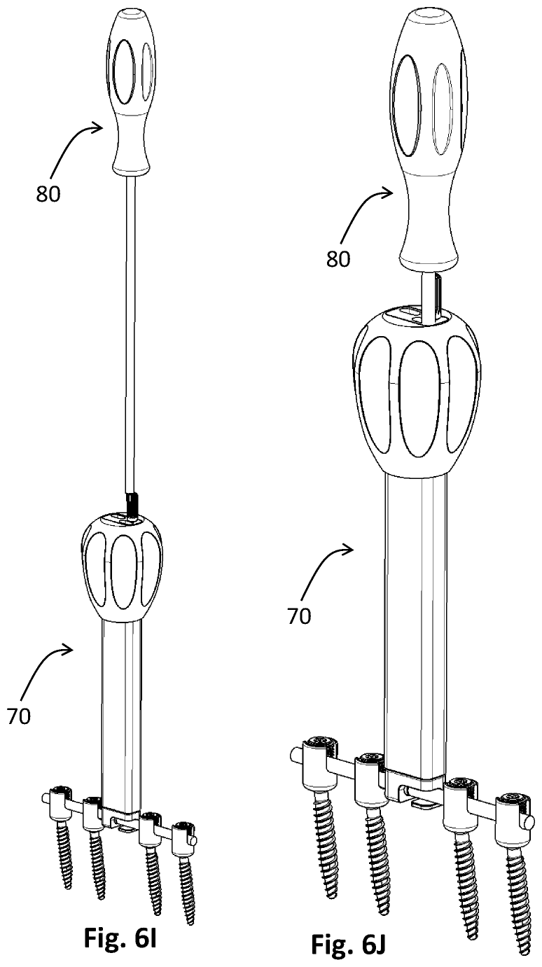

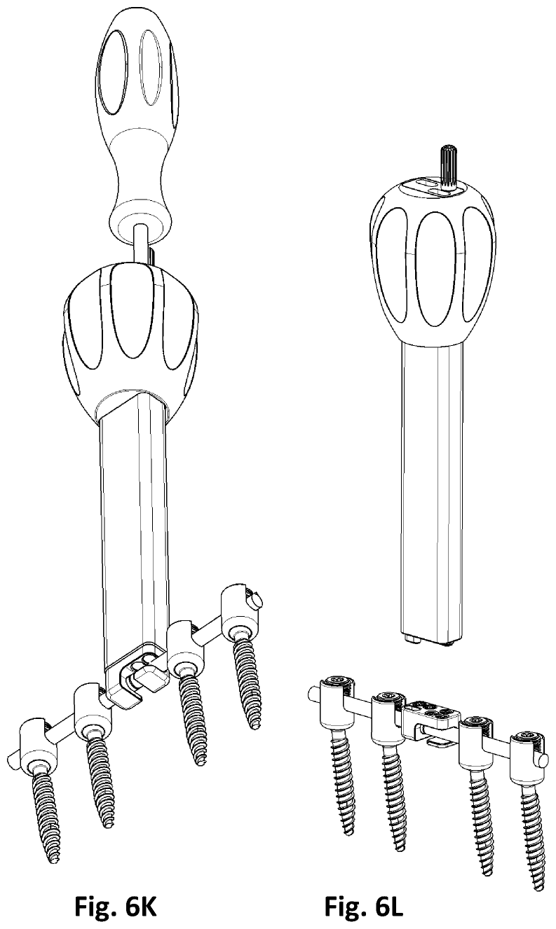

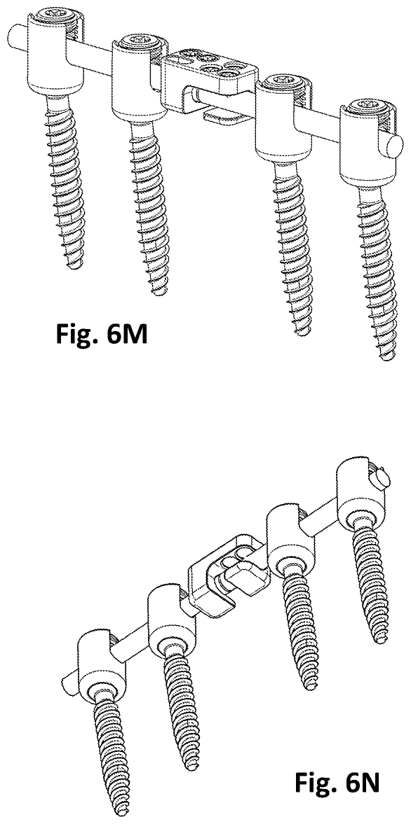

FIGS. 6A to 6N illustrate the steps of repairing a broken posterior spinal construct by using of a top-loading approach. For illustration purposes, the spinal column is not shown. FIGS. 6A and 6B depict a broken posterior spinal construct or system in place or in situ. Four bone fasteners are shown, namely a first bone fastener 110a, a second bone fastener 110b, a third bone fastener 110c, and a fourth bone fastener 110d. The bone fasteners are depicted as state-of-the-art pedicle screws, most commonly used for stabilising the posterior spine. The present example illustrates the repair of the posterior spinal system between the second and third bone fasteners (or spinal column vertebra), at the fracture zone or region 102 defining a first rod portion or fragment 100 and a second rod portion or fragment 101. It is to be noted that in the present example description, the first and second rod portions are shifted over a distance ‘x’ in relation to each other. FIGS. 6A and 6B also show the insertion instrument 70, which is connected to the rod connector assembly 60. In this example, the rod connector assembly includes the rod connector 11, including engaged rod fasteners 50a to 50d, as depicted in FIGS. 1A to 1C. It is to be noted that the rod fasteners are engaged in such a manner that the passage 18 is free to receive the broken rod portions.

FIGS. 6C and 6D depict the rod connector assembly 60 when in contact with at least one of the rod portions. The pocket 31 provides a clearance for the rods to pass through and to rest against the third passage top wall 22c. In this position, the passage including the passage axis are oriented at an acute angle with respect to the longitudinal axis of the rod portions. FIGS. 6E and 6F show the turning of the rod connector assembly using the inserter instrument around the axis A4. The axis A4 is a virtual turning axis extending through the implant and instrument assembly, and is oriented substantially orthogonally to the passage 18 including the passage axis. FIGS. 6E and 6F further show the moment, where the side seats 24a and 24b come in contact with the shifted rod portions. FIGS. 6G and 6H show the realigning of the rod portions using the rod connector assembly. By forceful turning, the portions are brought to their original position before breakage. Now, the side seats 24a and 24b are engaged with the rod portions.

FIGS. 6I, 6J and 6K show the engagement of a locking or tightening tool 80, such as a screw driver. The implant inserter includes at least one guiding channel extending through the inserter shaft, providing an aid for precisely engaging the locking tool driving tip into the drives of the rod fasteners. In a next step, all rod fasteners 50a to 50d are tightened to rigidly lock the rod portions within the rod connector 10. FIG. 6L shows the disengagement of the inserter instrument from the rod connector assembly 60. FIGS. 6M and 6N show the repaired posterior spinal stabilisation system.

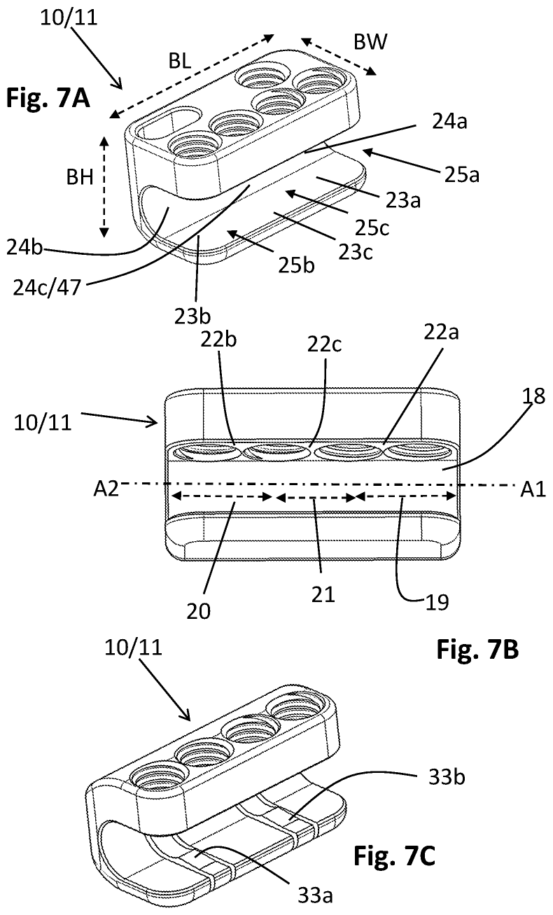

Referring to FIGS. 7A to 7C, an alternative design of the rod connector 10 is shown in detail. The rod connector 10 comprises a rod connector body 11 having a body length BL, a body width BW and a body height BH, where the body width BW and body height BH define a first body end 12, and a second body end 13, the body length BL and the body height BH define a first body side 14 and second body side 15, and the body length BL and the body width BW define a body top side 16 and a body bottom side 17 in a similar manner to the design of FIG. 2A. It is to be understood that for illustrative purposes, in FIGS. 7A and 7B, the connector body is depicted being substantially block shaped with rounded edges. Alternatively, the body may have for example a cylindrical, tubular, oblong, or triangular shape, or it may be differently shaped, but it would still have an average width, length and height defining a physical or virtual first body end 12, a second body end 13, a first body side 14, a second body side 15, a body top side 16 and a body bottom side 17. FIG. 7C depicts a more rounded version of the rod connector.

The rod connector body 11 further comprises a rod receiving passage 18 extending between the first body end 12 and the second body end 13, where the passage 18 comprises a first passage end section 19, a second passage end section 20 and a passage middle section 21 located between the first and second passage end sections. These different passage sections are thus located longitudinally along the passage. The first passage end section is at least open or extending into the first body end 12 and into the first body side 14. In a similar manner, the second passage end section is at least open or extending into the second body end 13 and the first body side 14. Furthermore, the passage middle section 21 is also open towards the first body side 14. In other words, in this specific design of the rod connector, all the passage sections are open towards the first side. The first passage end section comprises a first passage top wall 22a, a first and substantially opposite first passage bottom seat 23a, and a first passage side seat 24a extending between the top wall and the bottom seat. Opposite to the first passage side seat 24a, a first passage side entrance 25a is present. The second passage end section comprises a second passage top wall 22b, a second and substantially opposite second passage bottom seat 23b, and a second passage side seat 24b, extending between the top wall and the bottom seat. Opposite to the second passage side seat 24b a second passage side entrance 25b is present. The passage middle section comprises a third passage top wall 22c, a third and substantially opposite third passage bottom seat 23c, and a middle passage side wall 47 extending between the top wall and the bottom seat. Opposite to the third passage side seat 24b, a third passage side entrance 25c is present. The passage sections are sized and shaped to capture the rod portions as described later. In this example, the side wall 47 forms the side seat 24c. Furthermore, the first passage end section and the second passage end section define a first passage central axis A1, and a second passage central axis A2, respectively. These axes are aligned and they coincide with each other, or they intersect each other. The passage side seats in this example (as also in the other example explained in the present description) have a rounded surface, which has a shape, which is a complementary to the shape of the surface of the rod portions.

The body top side 16 comprises at least one locking hole 26a, 26b, 26c, 26d extending from the body top side into or over the passage. The locking holes are sized and shaped for engaging with at least one rod fastener 50a to 50d. According to one example, at least one of the locking holes comprises an internal thread feature, while at least one of the rod fasteners comprises an external thread feature, or vice versa. Furthermore, the body top side 16 may comprise at least one first instrument attachment means 28a, 28b for connecting to an implant inserter 70. Alternatively (not depicted) at least one of the first body end 12, second body end 13, first body side 14, second body side 15, and body bottom side 17 comprises at least one instrument attachment means 28a, 28b.

Referring to FIG. 7C, the rod connector 10 according to this example comprises at least one flexible or compliant finger 33a, 33b intended to provide initial stability between the rod connector and the rods before tightening one of the rod fasteners 50a to 50d. The compliant finger will deflect away or outwardly when a rod is pressed against the finger 33a, 33b, and will deflect back or inwardly to its initial or rest position when the rod has passed, and sits in its end position in the passage. In the present example, the finger encompasses the rod over a greater distance of the circumference of the rod than the passage itself.

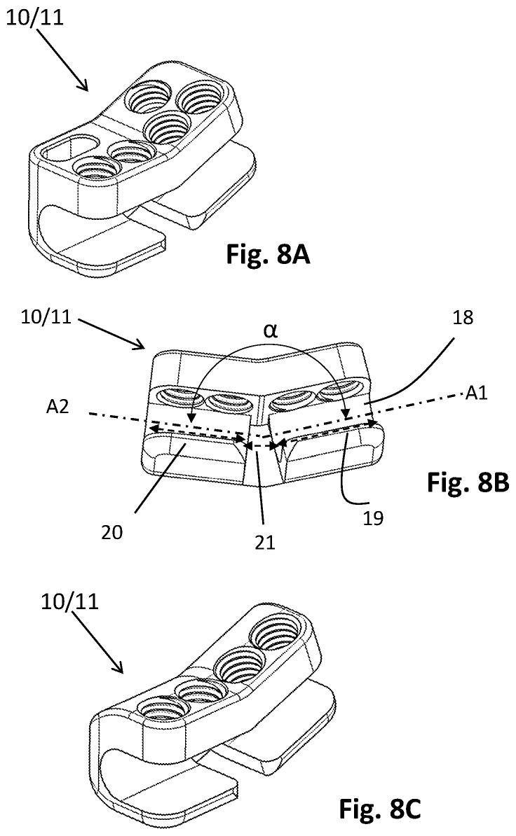

FIGS. 8A to 8C show an alternative shape of the rod connector, addressing the need to repair a fractured rod, broken in a hard bend region. In this case the first passage end section 19 and the second passage end section 20 are oriented at an obtuse angle α relative to the other. The curvature considerations presented above also apply to the design of FIGS. 8A to 8C.

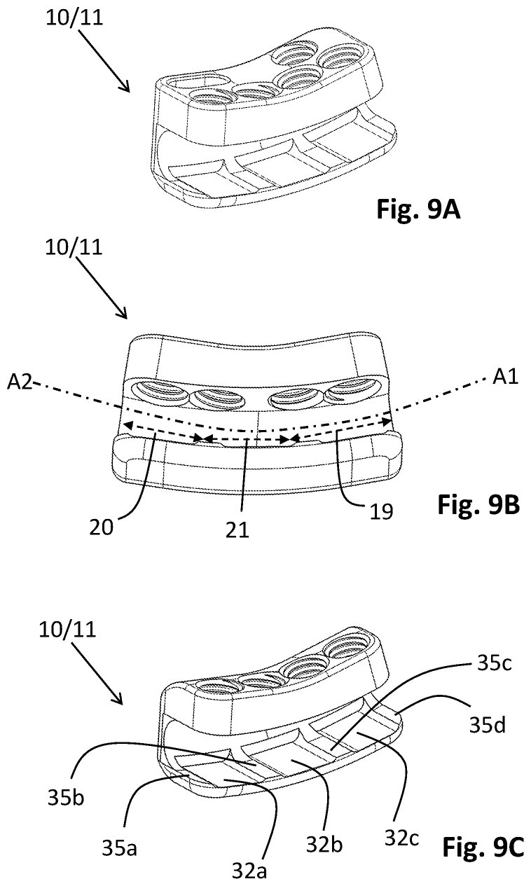

FIGS. 9A to 9C show an alternative shape of the rod connector, addressing the need to repair a fractured rod, broken in a soft bend or curved region. In this case the first passage end section 19 and second the passage end section 20 have a curvature of equal radius as the rod they are intended to receive within the passage sections. The rod connectors as depicted in FIGS. 9A to 9C are sized and shaped for a so-called side-loading approach. The curvature considerations presented above also apply to the design of FIGS. 9A to 9C.

The rod connector as depicted in FIGS. 9A to 9C further comprises at least one bottom recess 32a, 32b, 32c, which all extend from the bottom seat 23 towards the body bottom side 17, and define passage bottom seat partitions 35a, 35b, 35c, 35d. In the present example, three bottom recesses 32a, 32b, 32c define four passage bottom seat partitions 35a, 35b, 35c, 35d so that one passage bottom seat partition 35b is defined between a first bottom recess 32a and a second bottom recess 32b, while another passage bottom seat partition 35c is defined between the second bottom recess 32b and a third bottom recess 32c. In the cases, where at least one of the rod curvatures is not completely congruent to the passage curvatures, the rod will engage against for example two bottom seat partitions, and can still be locked rigidly by tightening the rod fasteners. The presence of the bottom recesses allows for larger shape differences between the passage and the rod.

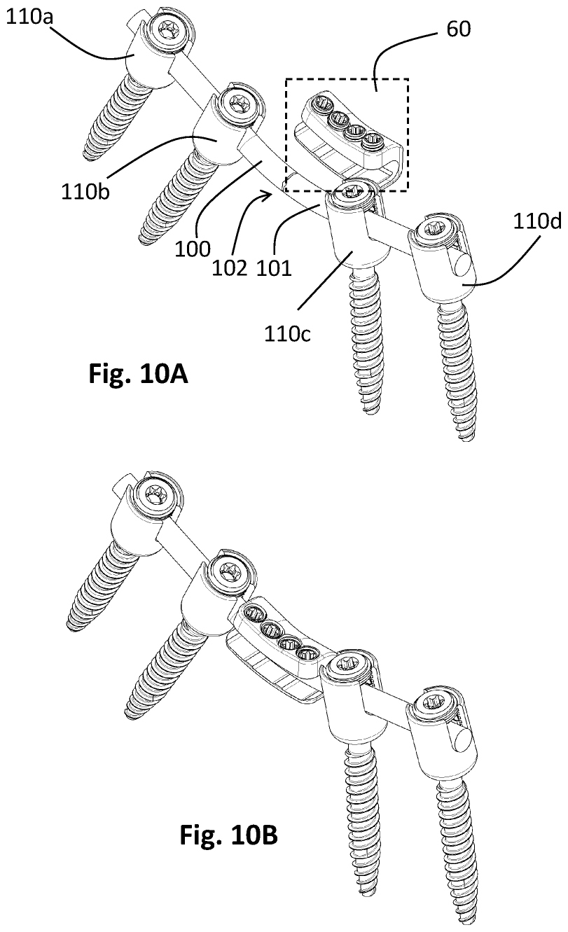

FIGS. 10A and 10B illustrate the steps of repairing a broken posterior spinal construct by means of a side-loading approach. For illustration purposes, the spinal column is not shown. FIGS. 10A and 10B show a broken posterior spinal construct or system in place or in situ. Four bone fasteners are shown, namely a first bone fastener 110a, a second bone fastener 110b, a third bone fastener 110c, and a fourth bone fastener 110d. The bone fasteners are depicted as state-of-the-art pedicle screws, most commonly used for stabilising the posterior spine. The present example illustrates the repair of the posterior spinal system between the second and third bone fasteners (or spinal column vertebra), at the fracture zone 102, where the first and second rod portions 100, 101 are present. As depicted, the rod connector assembly 60 is placed from the side over the broken rod portions. After bringing the rod connector in place, the rod fasteners are tightened to rigidly fixate the whole construct.

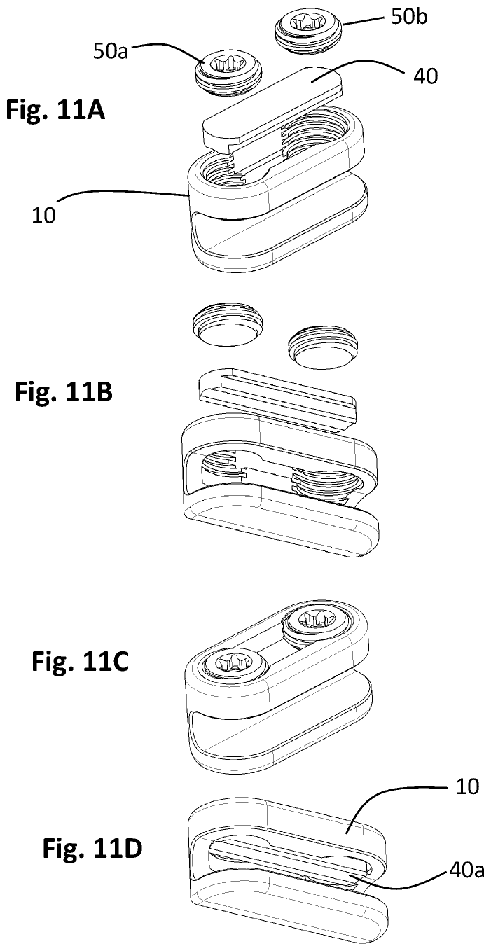

Referring to FIGS. 11A to 11D an alternative design of the side-loading rod connector is shown. The rod connector assembly 60 comprises at least one intermediate insert 40 sized and shaped to engage against at least one of the rod portions 100, 101. The insert transfers the tightening forces created by at least one of the rod fasteners 50a, 50b to the rod portions, and so rigidly locks the rod portions within the rod connector. In this case, the insert is provided between the passage and the body top side 16, and thus between the passage and the rod fasteners. The insert 40 as shown is a longitudinal element that in this example extends longitudinally beneath the body top side 16 between at least two locking holes. Alternatively, the insert and the rod connector body can form a monolithic component, so that the intermediate insert is configured as at least one compliant structure, and configured to undergo elastic deformation.

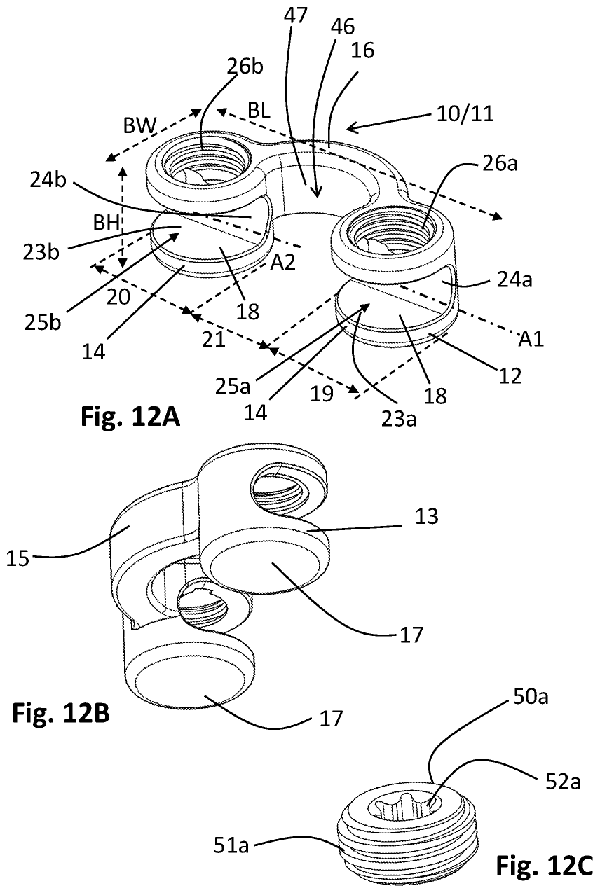

Another situation of rod breakage of an in situ spinal construct is characterised by very short and long fractured rod ends between the bone fasteners, where the short end is too short for attachment of a connector thereto. The connector must be connected over the bone fastener at the opposite side of the bone fastener. Referring to FIGS. 12A to 12C, another embodiment of the rod connector is shown addressing this scenario. The middle section 21 of the rod connector is sized and shaped to circumvent, bypass or encompass the cylindrical top portion or head of a bone fastener. The rod connector 10 comprises a rod connector body 11 and defines the first body end 12, second body end 13, first body side 14, second body side 15, body top side 16, and body bottom side 17 in a similar manner as they were defined in connection with FIG. 2A, for instance. The connector body 11 further comprises a rod receiving passage 18 extending from the first body first end 12 to the second body end 13, where the passage 18 is divided into a first passage end section 19, a second passage end section 20, and a passage middle section 21. The first passage end section is at least open or extending into the first body end 12 and into the first body side 14. The second passage end section is at least open or extending into the second body end 13 and into the first body side 14. The middle section 21 is also open towards the first body side 14. A curved or bent connecting portion (which may be considered the middle section or a portion of it) is provided for connecting the first passage end section to the second passage end section. The connecting portion thus forms an arch to surround or partially surround a tip of a bone fastener as explained later.

The first passage end section comprises a first passage top wall 22a, a first and substantially opposite first passage bottom seat 23a, and a first passage side seat 24a extending between the top wall and the bottom seat. Opposite to the first passage side seat 24a, a first passage side entrance 25a is present. In a similar manner, the second passage end section comprises a second passage top wall 22b, a second and substantially opposite second passage bottom seat 23b, and a second passage side seat 24b extending between the top wall and the bottom seat. Opposite to the second passage side seat 24b, a second passage side entrance 25b is present. The passage sections are sized and shaped to capture the rod portions as described later. The middle section is sized and shaped as a second pocket 46 starting at the body first side 14 and the body bottom side 17 and has a pocket length PL measured along the direction of the body length BL. The second pocket may extend along the full body height, or it may instead extend from the body bottom side only along a portion of the body height towards the top side 16. The middle section may comprise a third passage side seat or wall 24c, and it may also comprise a passage top wall 22c. Furthermore, the first passage end section, second passage end section and middle section define at least two central axes A1, A2, which may be aligned and coincide with each other or they may intersect each other.

The body top side 16 comprises at least one locking hole 26a, 26b, 26c, 26d, extending from the body top side into or over the passage. The locking holes 26 are sized and shaped for engaging with at least one rod fastener 50a to 50d. According to one example, at least one of the locking holes is configured as an internal thread feature, while at least one of the rod fasteners comprises an external thread feature, or vice versa. Furthermore, the body top side 16 may comprise at least one first instrument attachment means 28a, 28b for coupling to an implant inserter 70. Alternatively, (not depicted) at least one of the first body end 12, second body end 13, first body side 14, second body side 15, and body bottom side 17 comprises at least one instrument attachment means 28a, 28b.

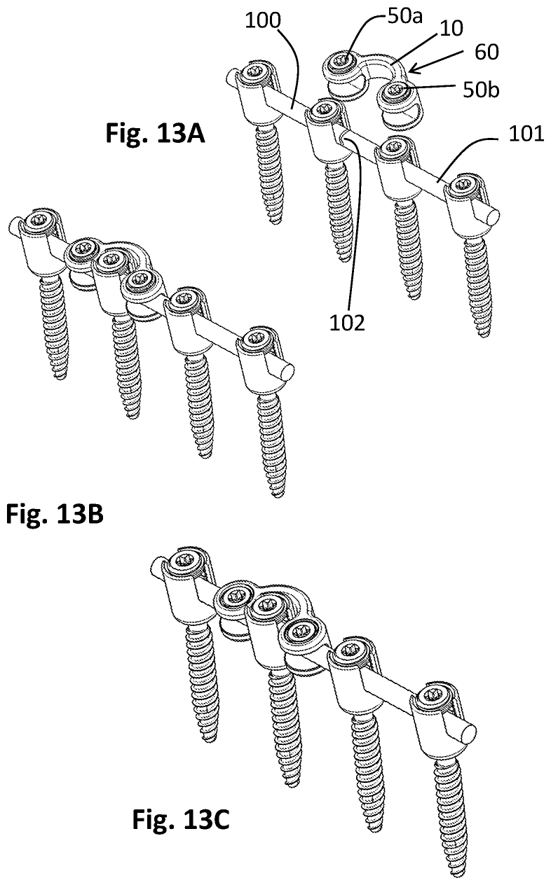

FIGS. 13A to 13C depict a broken posterior spinal construct or system in place or in situ. The system comprises four bone fasteners. A first rod portion 100 and a second rod portion 101 are separated by a fracture zone 102. It is to be noted that in this example, the fracture zone 102 is directly adjacent to a bone fastener. As depicted, the rod connector assembly 60 is placed from the side, in a horizontal orientation over the broken rod portions. The head of the bone fasteners may now rest against the middle passage side wall 47, while the first rod portion may rest against the walls or seats of the first passage end section, and the second rod portion may rest against the walls or seats of the second passage end section. After bringing the rod connector in place, the bone fasteners are tightened to rigidly fixate the whole construct.

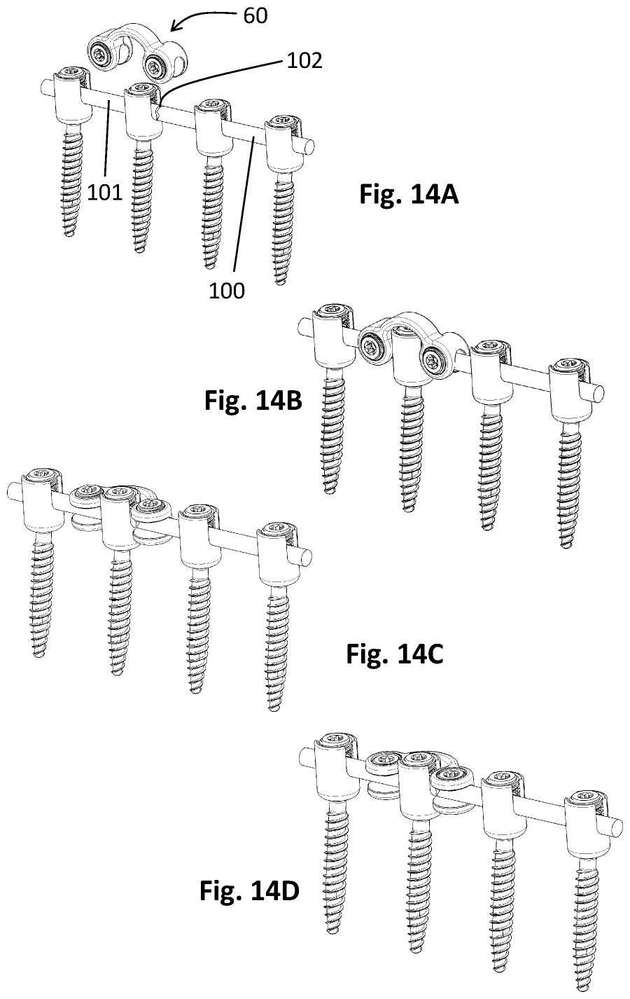

FIGS. 14A to 14D depict an alternative surgical technique, connecting the rod portions using a top-loading approach. As depicted, the rod connector assembly 60 is placed in a vertical orientation over the broken rod portions. After bringing the rod connector assembly in place, the rod connector assembly is turned over for example approximately 90°, and brought in a horizontal orientation. Afterwards the bone fasteners are tightened to rigidly fixate the whole construct.

While the invention has been illustrated and described in detail in the drawings and foregoing description, such illustration and description are to be considered illustrative or exemplary and not restrictive, the invention being not limited to the disclosed embodiments. Other embodiments and variants are understood, and can be achieved by those skilled in the art when carrying out the claimed invention, based on a study of the drawings, the disclosure and the appended claims. New embodiments or variants may be obtained by combining any of the above teachings.

In the claims, the word “comprising” or “including” does not exclude other elements or steps, and the indefinite article “a” or “an” does not exclude a plurality. The mere fact that different features are recited in mutually different dependent claims does not indicate that a combination of these features cannot be advantageously used. Any reference signs in the claims should not be construed as limiting the scope of the invention.

1. A spinal rod connector assembly for connecting and axially aligning two in situ spinal posterior rod portions, the spinal rod connector assembly comprising:
one or more rod fasteners; and
a rod connector having:
a first connector end,
a second, opposite connector end,
a first connector side,
a second, opposite connector side,
a connector top side,
an opposite connector bottom side and
a rod receiving passage extending between the first connector end and the second connector end, the passage including:
a first passage end section,
a second passage end section, and
a passage middle section between the first and second passage end sections,
wherein:
the first passage end section includes a first passage top wall, a first passage bottom seat opposite to the first passage top wall, and a first passage side seat between the first passage top wall and the first passage bottom seat, a first passage entrance being provided opposite to the first passage side seat, the first passage end section defining a first passage central axis,
the second passage end section includes a second passage top wall, a second passage bottom seat opposite to the second passage top wall, and a second passage side seat between the second passage top wall and the second passage bottom seat, a second passage entrance being provided opposite to the second passage side seat, the second passage end section defining a second passage central axis aligned with, or intersecting the first passage central axis,
the passage middle section includes at least a third passage top wall and/or a third passage side wall, a third passage side entrance being provided adjacent to the first passage side entrance and/or the second passage side entrance, and
the rod connector includes one or more locking holes, extending from the connector top side into or towards the rod receiving passage, the one or more locking holes being sized and shaped for engaging with the one or more rod fasteners.

2. The spinal rod connector assembly according to claim 1, wherein the first and second passage side seats are directed in opposite directions.

3. The spinal rod connector assembly according to claim 1, wherein the first and second passage side seats are directed in the same direction.

4. The spinal rod connector assembly according to claim 1, wherein:
the passage middle section comprises:
a third passage side seat between the first and second passage side seats, and
a third passage bottom seat between the first and second passage bottom seats, and
the third passage side seat is directed in the same direction or substantially in the same direction as the first and second passage side seats.

5. The spinal rod connector assembly according to claim 1, wherein at least one of the first and second passage central axes is curved.

6. The spinal rod connector assembly according to claim 1, wherein the one or more rod fasteners are sized and shaped for direct engagement with the first and/or second rod portions.

7. The spinal rod connector assembly according to claim 1, wherein:
the spinal rod connector assembly comprises an insert between the passage and
the connector top side, and wherein the insert is configured to directly engage with the first and/or second rod portions.

8. The spinal rod connector assembly according to claim 1, wherein at least one of the first connector end, second connector end, first connector side, second connector side, connector top side, and connector bottom side comprises one or more instrument attachment means for coupling the rod connector to an implant inserter.

9. The spinal rod connector assembly according to claim 1, wherein the passage middle section is sized and shaped as a pocket extending from the first connector side to the second connector side, and being open at the connector bottom side.

10. The spinal rod connector assembly according to claim 1, wherein at least one of the passage bottom seats comprises a recess extending towards the connector bottom side forming a clearance between at least two adjacent passage bottom seat partitions.

11. The spinal rod connector assembly according to claim 1, wherein:
at least one of the locking holes comprises an internal thread feature, and
at least one of the rod fasteners comprises an external thread feature, or vice versa.

12. The spinal rod connector assembly according to claim 1, wherein the first and second passage central axes are oriented at an obtuse angle with respect to each other.

13. The spinal rod connector assembly according to claim 1, wherein the first, second and/or third passage side seat(s) has/have a shape which is complementary to a shape of a respective rod portion with which the respective passage side seat is configured to come in contact.

14. The spinal rod connector assembly according to claim 1, wherein the first, second and/or third passage bottom seat(s), and/or the first and/or second passage top wall(s) has/have a flat or substantially flat surface.

15. The spinal rod connector assembly according to claim 1, wherein a respective bottom seat surface is parallel to its opposite top wall surface.

16. The spinal rod connector assembly according to claim 1, wherein the third passage side wall has a curvature to partially surround a tip of a bone fastener.

17. A kit comprising at least a first spinal rod connector assembly and a second spinal rod connector assembly according to claim 1, wherein the first rod connector of the first spinal rod connector assembly is sized and/or shaped differently with respect to the second rod connector of the second spinal rod connector assembly.

18. The kit according to claim 17, wherein the first rod connector of the first spinal rod connector assembly, and the second rod connector of the second spinal rod connector assembly are mirrored elements.

19. The kit according to claim 17, wherein:
the first passage central axis of the first spinal rod connector assembly is oriented differently from the first passage central axis of the second spinal rod connector assembly, and/or
the second passage central axis of the first spinal rod connector assembly is oriented differently from the second passage central axis of the second spinal rod connector assembly.

20. A method for connecting and axially aligning two in situ spinal posterior rod portions by using the spinal rod connector assembly according to claim 1, the method comprising:
placing the rod connector or the rod connector assembly over at least one of the two rod portions;
engaging the passage middle section with the two rod portions;
turning the rod connector over an angle in the range of 60° to 120° around an insertion instrument central axis of an insertion instrument used for the turning, leading to an engagement of the two rod portions with at least two of the passage side seats; and
tightening the one or more rod fasteners by using a tightening tool.
